(19) United States
(12) Patent Application Publication
(10) Pub. No.: US 2019/0035505 A1
(43) Pub. Date: Jan. 31, 2019

(54) INTELLIGENT TRIAGE SERVER, TERMINAL AND SYSTEM BASED ON MEDICAL KNOWLEDGE BASE (MKB)

(71) Applicant: BOE TECHNOLOGY GROUP CO., LTD., Beijing (CN)

(72) Inventor: Hui LI, Beijing (CN)

(21) Appl. No.: 15/984,859

(22) Filed: May 21, 2018

(30) Foreign Application Priority Data
Jul. 31, 2017 (CN) 201710642039.7

(51) Int. Cl. G16H 50/20; G16H 50/70; G06F 17/27

(52) U.S. Cl. CPC G16H 50/20; G06F 17/2785; G16H 50/70

(57) ABSTRACT

An intelligent triage server based on a medical knowledge base (MKB), including: a processor and a memory, the memory is for storing computer program instructions, wherein, the computer program instructions, when being executed by the processor, perform the processes of: receiving a retrieval request from an intelligent triage terminal; performing entity recognition and type analysis on the retrieval request; retrieving one or more pieces of knowledge by utilization of the index of the MKB according to the entities recognized and the types analyzed; sorting the one or more pieces of knowledge retrieved, and regarding the prior knowledge as analysis results; and transmitting the analysis results obtained to the intelligent triage terminal.

### TECHNICAL FIELD

The embodiments in the present disclosure relates to an intelligent triage server, a terminal based on medical knowledge base (MKB) and a system comprising the server and the terminal.

### BACKGROUND

China's medical industry currently faces many problems, including: the patients are lack of medical treatment and health knowledge; the patients cannot correctly assess their own symptoms, do not know when to go to the hospital, do not know which department to register with, often register wrong department, and go to a large hospital for minor illnesses, thus reducing the medical efficiency and wasting the medical resources; the physicians have a heavy workload and do not have enough time to answer all the patients' questions, causing a strained doctor-patient relationship; and the medical resources are uneven and in shortage, leading to medical difficulty, etc.

On the whole, medical science is knowledge-driven science. If we collect adequately abundant and reliable knowledge from multiple dimensions, the knowledge can play a huge role in assisting decision-making and free medical personnel from complicated and repetitive tasks, and then the medical personnel can go to do more creative things. Most of the traditional diagnostic thinking use a human-edited MKB. The MKB is small in scale, lags behind in the updating of knowledge and information, adopts the structure of a decision tree, and will soon reach a bottleneck in diagnostic effect. The conventional MKB generally includes knowledge items. These knowledge items are usually in free text format, which severely affects the retrieval efficiency.

### SUMMARY

According to at least one embodiment of this disclosure, an intelligent triage server based on a medical knowledge base (MKB) is provided, comprising: a processor and a memory, the memory is for storing computer program instructions, wherein, the computer program instructions, when being executed by the processor, perform the processes of: receiving a retrieval request from an intelligent triage terminal; performing entity recognition and type analysis on the retrieval request; retrieving one or more pieces of knowledge by utilization of the index of the MKB according to the entities recognized and the types analyzed; sorting the one or more pieces of knowledge retrieved, and regarding the prior knowledge as analysis results; and transmitting the analysis results obtained to the intelligent triage terminal.

For example, sorting the one or more pieces of knowledge retrieved comprises: sorting the one or more pieces of knowledge retrieved according to the degree of confidence from high to low.

For example, performing entity recognition and type analysis on the retrieval request comprises: performing entity recognition via at least one method or a combination of hidden Markov model (HMM), maximum entropy model (MaxEnt), support vector machine (SVM) and conditional random field (CRF).

For example, performing entity recognition and type analysis on the retrieval request comprises: performing entity recognition via bootstrapping method.

For example, performing entity recognition and type analysis on the retrieval request comprises: performing type analysis via at least one classifier or a combination of decision tree, random forest, logistic regression, gradient boost and SVM.

For example, the recognized entities comprise at least one of the follows: symptom entity, time entity, disease entity, personal information entity and medical site entity.

For example, the types comprise at least one of the follows: pre-diagnosis type, triage type and healthcare type.

For example, the MKB comprises structural data.

For example, the computer program instructions, when being executed by the processor, further perform the processes of: creating or updating the MKB.

For example, wherein, creating or updating the MKB comprises: extracting entities, attributes, and relationships, from semi-structured data and unstructured data in a data source, as knowledge; fusing the extracted knowledge; and calculating the degree of confidence of the fused knowledge, and storing the knowledge, of which the degree of confidence exceeds the threshold, into the MKB.

For example, creating or updating the MKB further comprises: after fusing the extracted knowledge, obtaining implicit knowledge by the reasoning of the fused knowledge; and calculating the degree of confidence of the obtained implicit knowledge, and storing the implicit knowledge, of which the degree of confidence exceeds the threshold, into the MKB as well.

For example, extracting entities, attributes from semi-structured data and unstructured data comprises: extracting entities, attributes from semi-structured data and unstructured data by any one of supervised algorithm, semi-supervised algorithm, unsupervised algorithm and distant supervision algorithm.

For example, extracting relationships from semi-structured data and unstructured data comprises: extracting relationships from the semi-structured data and the unstructured data by supervised algorithm.

For example, fusing the extracted knowledge comprises: integrating the knowledge extracted from the semi-structured data and the unstructured data, in which the integration includes entity alignment, attribute value decision and relationship building.

According to at least one embodiment of this disclosure, a intelligent triage terminal is provided, which is connected to the intelligent triage server based on the MKB, comprising: a processor and a memory, the memory is for storing computer program instructions, wherein, the computer program instructions, when being executed by the processor, perform the processes of: receiving a retrieval request inputted by an user; transmitting the retrieval request to the intelligent triage server; receiving retrieval results obtained by the intelligent triage server from the MKB on the basis of the retrieval request; displaying the retrieval results to the user.

For example, displaying the retrieval results to the user comprises: displaying the retrieval results to the user based on a sequence specified by the user.

For example, the retrieval results is sorted according to the probability of possible diseases from high to low by the intelligent triage terminal, items of probable diseases is displayed on a display region of the intelligent triage terminal.

For example, both of the retrieval request of the user and the retrieval results comprises a form of natural language.

According to at least one embodiment of this disclosure, an intelligent triage system is provided, comprising: the intelligent triage server based on the MKB according to the previous embodiments and an intelligent triage terminal, wherein, the intelligent triage terminal is connected to the intelligent triage server based on the MKB, comprising: a processor and a memory, the memory is for storing computer program instructions, wherein, the computer program instructions, when being executed by the processor, perform the processes of: receiving a retrieval request inputted by an user; transmitting the retrieval request to the intelligent triage server; receiving retrieval results obtained by the intelligent triage server from the MKB on the basis of the retrieval request; displaying the retrieval results to the user.

According to at least one embodiment of this disclosure, a intelligent triage system is provided, comprising: the intelligent triage server based on the MKB according to the previous embodiments and an intelligent triage terminal, wherein, the intelligent triage terminal is connected to the intelligent triage server based on the MKB, comprising: a processor and a memory, the memory is for storing computer program instructions, wherein, the computer program instructions, when being executed by the processor, perform the processes of: receiving a retrieval request inputted by an user; transmitting the retrieval request to the intelligent triage server; receiving retrieval results obtained by the intelligent triage server from the MKB on the basis of the retrieval request; displaying the retrieval results to the user.

### BRIEF DESCRIPTION OF THE DRAWINGS

In order to make the skilled in the art knows about this disclosure better, the embodiments of this disclosure are described specifically referring the drawings, but the description should not be regarded as the limitation of this disclosure. The characteristics, benefits, techniques and feasibility of the example embodiments will be described referring the drawings in the following. In the drawings, same drawing references indicate same elements. And also in the figures:

FIG. 1 is a schematic diagram illustrating the main principles of an intelligent triage system based on an MKB provided by the first embodiment of the present disclosure;

FIG. 2 is a block diagram of an intelligent triage system 200 based on an MKB provided by the second embodiment of the present disclosure, in which a block 201 indicates an intelligent triage terminal in the intelligent triage system; a block 202 indicates an intelligent triage server in the intelligent triage system; a block 203 indicates a data source;

FIG. 3 is layered architecture diagram of an intelligent triage system based on an MKB provided by the third embodiment of the present disclosure;

FIG. 4 is a schematic diagram illustrating fused medical knowledge in the fourth embodiment of the present disclosure; and

FIGS. 5(a) to 5(e) are schematic diagrams of an interface of an intelligent triage terminal 201 based on an MKB provided by the fifth embodiment of the present disclosure.

### DETAILED DESCRIPTION

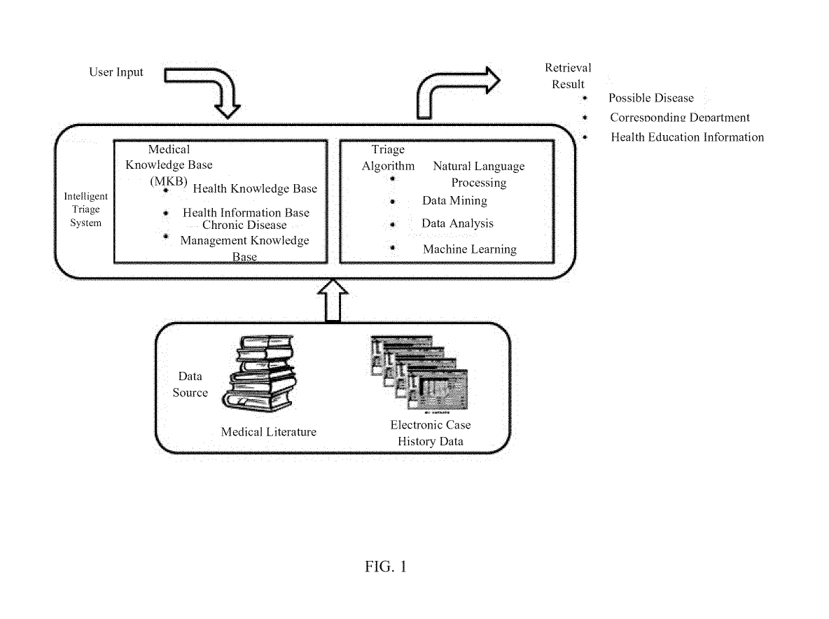

Detailed description will be given below to the present disclosure with reference to the accompanying drawings. FIG. 1 is a schematic diagram illustrating the main principles of an intelligent triage system based on an MKB provided by the first embodiment of the present disclosure. As illustrated in FIG. 1, the intelligent triage system is communicated with and linked to a data source. The data source includes unstructured text data such as medical literature and semi-structured data such as electronic case history. The intelligent triage system receives user input; the input includes the user's personal information, retrieval request and the like in the form of natural language; the intelligent triage system outputs a retrieval result; and the retrieval result may include at least one of disease that the user may suffer from (the user's pre-diagnosis type retrieval request), corresponding department (the user's triage type retrieval request), or health education information (the user's health care type retrieval request).

For instance, the intelligent triage system may comprise: a processor; and a memory, in which computer executable instructions are stored in the memory; when the computer executable instructions are executed by the processor, a triage algorithm based on natural language processing and machine learning technology is executed. The processor includes general processor (such as Central Processing Unit) or specified processor (such as programmable Logic Circuit). And for example, the triage algorithm includes: performing entity recognition and type analysis on the retrieval request inputted by the user; retrieving one or more pieces of knowledge in the MKB by utilization of the index of the MKB according to recognized entities and analyzed types; and regarding the retrieved knowledge as retrieval results. In another example, selecting those knowledge with high degree of confidence as retrieval results, and providing the retrieval results to the user. For example, the triage algorithm may further include: mining, analyzing and extracting data from the data source, optionally fusing the extracted knowledge, and creating and/or updating the MKB. It should be noted that: although FIG. 1 takes the case that the MKB is included in the intelligent triage system as an example, the MKB is not necessarily stored into the memory, as long as the MKB can be called by the intelligent triage system; and in addition, the system not only can use the MKB created and/or updated by the system but also can share the MKB with other systems. Thus, the triage algorithm integrates the functions such as natural language processing, data mining, data analysis and machine learning (e.g., deep learning). For example, the MKB may include at least one of health knowledge base, health information base or chronic disease management knowledge base.

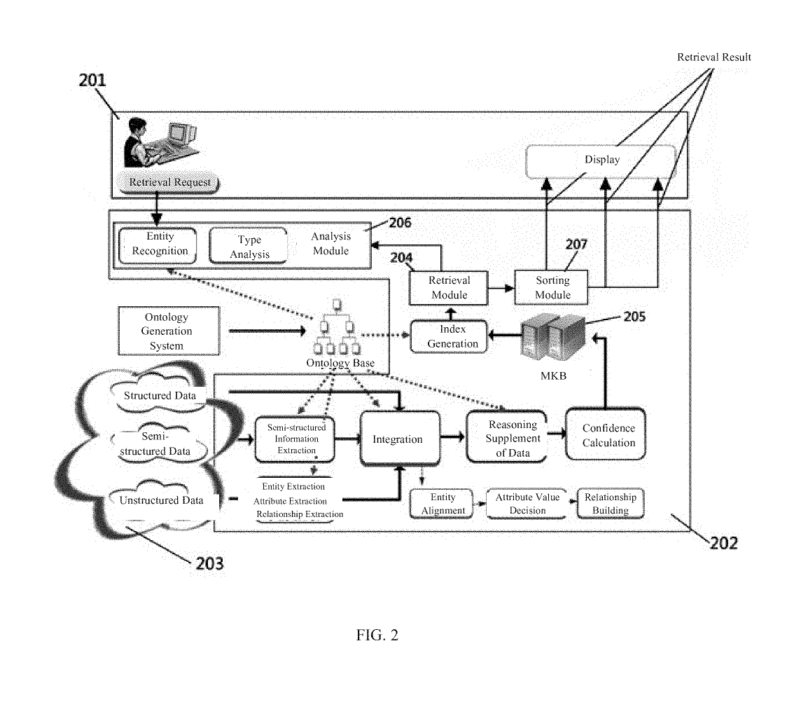

FIG. 2 is a block diagram of an intelligent triage system 200 based on an MKB provided by the second embodiment of the present disclosure, in which a block 201 indicates an intelligent triage terminal in the intelligent triage system 200; a block 202 indicates an intelligent triage server in the intelligent triage system 200; and a block 203 indicates a data source. It should be noted that: in FIG. 2, square boxes indicate physical members (including software and/or hardware) such as various program modules, the intelligent triage terminal and an ontology base; round-corner boxes indicate the steps executed by these members, which are implemented by the execution of computer executable instructions stored on a memory (not shown in the figure) of the intelligent triage server 202; and a bubble box indicates various types of data.

As shown by the block 202 in FIG. 2, the intelligent triage server 202 is connected to the intelligent triage terminal and configured to receive the user's personal information (optional; the information may also be not included in the simplified version) and retrieval request from the intelligent triage terminal. The retrieval request refers to statement inputted into the intelligent triage terminal 201 by the user. The input manner, for instance, may include check in choice box, handwriting input or a combination thereof. For instance, the retrieval request may include personal information “2 years old girl” inputted by the user, symptom information “high fever at 39° C. for 2 hours” checked in the choice box, and question “go to the hospital or not” inputted by handwriting. Of course, the retrieval request may be generally simpler, for instance, only including the symptom information inputted by the user.

The intelligent triage server 202 obtains retrieval results for the retrieval request from the MKB 205, and transmits the retrieval results to the intelligent triage terminal 201, and then the intelligent triage terminal can display the retrieval results to the user.

For example, as shown in FIG. 2, the intelligent triage server 202 includes: a receiving module (not shown in the figure) configured to receive the retrieval request from the intelligent triage terminal 201; an analysis module 206 configured to perform entity recognition and type analysis on the retrieval request received by the receiving module; a retrieval module 204 configured to retrieve one or more pieces of knowledge in the MKB by utilization of the index of the MKB 205 according to the entities recognized and the types analyzed by the analysis module 206; a sorting module 207 configured to sort the one or more pieces of knowledge retrieved by the retrieval module 204 according to the degree of confidence from high to low, and transmit the prior knowledge, as the retrieval results, to an output module; and the output module (not shown in the figure) configured to transmit the obtained retrieval results to the intelligent triage terminal 201 for display.

Currently, the analysis module 206 may perform entity recognition by adoption of supervised method and semi-supervised method. For instance, the supervised method includes at least one method or a combination of HMM, MaxEnt, SVM and CRF. For instance, the semi-supervised method includes bootstrapping method. In general, the supervised algorithm needs to use labeled corpora for training before entity recognition.

For instance, entity recognition is performed on the retrieval request “1 year old girl”, “sudden high fever at 39° C. for 2 days continually” and “What disease? Go to hospital or not?”, and the recognized entities include: symptom type entities “sudden” and “high fever at 39° C.”; disease type entity “disease”; personal information type entities “1 year old” and “girl”; and medical site type entity “hospital”. The retrieval module 204 may retrieve the knowledge in the MKB 205 by utilization of the index of the MKB 205 according to the recognized entities. The index can be utilized to rapidly position these knowledge items including the entities, so as to greatly improve the retrieval efficiency. The knowledge item includes the entities as nodes, the attributes of the entities, and the relationships between the entities.

In order to choose knowledge that better fits the user's intentions from the retrieved knowledge including the recognized entities, type analysis may also be performed on the retrieval request, and those knowledge belonging to the analyzed types may be further selected from the knowledge retrieved according to the entities, so as to more precisely meet the user's needs. For example, the analysis module 206 may perform type analysis by utilization of at least one classifier or a combination of decision tree, random forest, logistic regression, gradient boost and SVM, and these classifiers must use labeled corpora for training before application. In general, the types include: pre-diagnosis type, for instance, possible disease and disease probability that the user wants to predict; triage type, for instance, what hospital to go and which department to register with that the user wants to know; and healthcare type, for instance, scientific knowledge of some disease, how to care and how to nurse that the user wants to get. Taking the retrieval request “1 year old girl”, “sudden high fever at 39° C. for 2 days continually” and “What disease? Go to hospital or not?” as an example, the classifiers are utilized to classify the retrieval request into pre-diagnosis type, triage type and healthcare type. Moreover, for instance, when the retrieval request does not include any sentence such as “What disease? Go to hospital or not?”, the user acquiescently needs to get all the three types of retrieval results, so as to avoid the missing of user needs.

Taking the retrieval request “1 year old girl”, “sudden high fever at 39° C. for 2 days continually” and “What disease? Go to hospital or not?” as an example, according to the recognized entities and the analyzed types, the finally obtained retrieval results are that:

possible disease: parascarlet

probability: 70%

recommended department: pediatric outpatient department

. . .

nursing recommendations: can stay home for observation; cooperated with physical cooling; taking antipyretics when the body temperature is greater than 38.5° C.; the fever is gone when rash appears usually after 3-5 days; the rash is pink or bright red spots; can go to the pediatric outpatient department if the fever has not gone when the rash appears at the time exceeding the time limit.

For instance, when the retrieval request does not include “go to hospital or not”, the type is determined to be pre-diagnosis type, and the finally obtained retrieval results only include the possible disease and the probability described above.

The sorting module 207 may be configured to calculate the degree of confidence of the one or more pieces of knowledge retrieved by the retrieval module 204, and sort the knowledge according to the degree of confidence from high to low. The degree of confidence may be calculated by utilization of numerous methods. For instance, the degree of confidence may be calculated by utilization of the frequency of the entities and the relationships in the knowledge appearing in the MKB 205, the number of sub-nodes in the knowledge item, or a combination thereof.

The data source 203 includes structured data, semi-structured data and unstructured data. The structured data is, for instance, the current MKB 205; the semi-structured data is, for instance, electronic case history; and the unstructured data is, for instance, text data from medical web pages, medical literature, medical guides, etc. The MKB 205 includes the structured data and may be created and updated by the following steps: extracting entities, attributes and relationships, from the semi-structured data and the unstructured data in the data source 203, as knowledge, fusing the extracted knowledge, calculating the degree of confidence of the fused knowledge, and storing the knowledge, of which the degree of confidence exceeds the threshold, into the MKB.

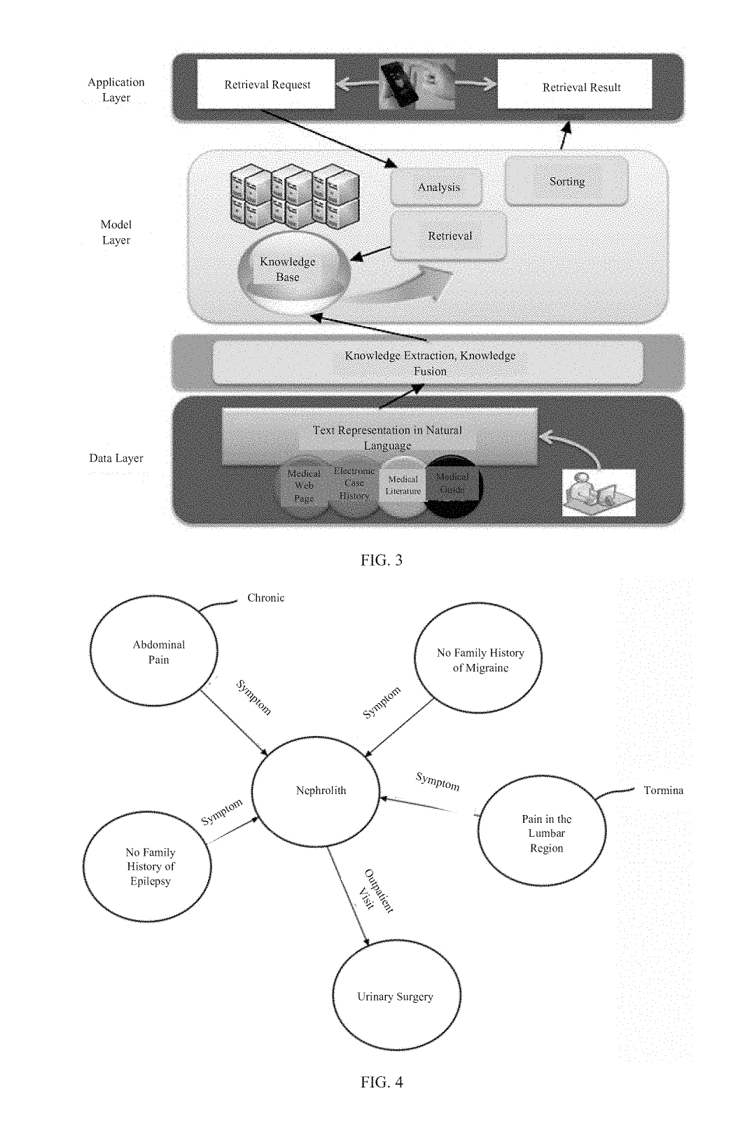

Detailed description will be given below to the steps of extraction and fusion with reference to FIG. 2. The entities, the attributes and the relationships are respectively extracted from the semi-structured data and the unstructured data to obtain the structured data (taken as the knowledge); the obtained structured data may be integrated; the integration of heterogeneous data is realized in the process of extracting and integrating the heterogeneous data; and the integrated data is also referred to as fused knowledge. Detailed description will be given below to the specific example of extraction with reference to FIG. 3, so no further description will be given here. The fusion of knowledge requires the data scrubbing of the conflict, duplicate redundancy and deviation of the entities, the attributes and the relationships, including entity alignment, attribute value decision, relationship building, etc.

For instance, taking entities “long-term intermittent stomach pain” and “abdominal pain for 1 week” as an example, the entities “stomach pain” and “abdominal pain” belong to the same entity and then may be aligned to be “abdominal pain”; and the attribute of the entity may be determined to be “chronic” from “long-term intermittent stomach pain”. For instance, the relationships among the entity “abdominal pain for 1 week” with the attribute “chronic”, an entity “no family history of migraine”, an entity “no family history of epilepsy”, an entity “pain in the lumbar region” with the attribute “tormina” and an entity “nephrolith” are that: the symptoms of “nephrolith” are usually “chronic abdominal pain”, “no family history of migraine”, “no family history of epilepsy” and “lumbar tormina”; and the relationship between the entity “nephrolith” and an entity “urinary surgery” is that: the patient having “nephrolith” must go to see a doctor of the “urinary surgery”. Thus, the two entities “long-term intermittent stomach pain” and “abdominal pain for 1 week” are aligned to be “abdominal pain” with the attribute “chronic”, and the relationship between the case that “abdominal pain” is generally one of the symptoms of “nephrolith” and the case that the patient must go to the “urinary surgery” department is created; and the fused knowledge is as shown in FIG. 4.

There is a considerable number of fused knowledge. If all the knowledge is stored into the MKB 205, the problems such as rapid and improper expansion of the MKB 205 and the occupancy of a memory space by low-value knowledge can be caused. In order to solve the problems, the degree of confidence of the fused knowledge may be calculated according to the method described above; a confidence threshold is set; the knowledge, of which the degree of confidence exceeds the threshold, is stored into the MKB 205; and then the memory space is For example left for those reliable knowledge, so as to ensure the storage efficiency of the intelligent triage server 202, the reasonable expansion of the MKB 205, and the quality of the knowledge in the MKB. For example, the step of creating and updating the MKB 205 further includes: after the step of fusing the extracted knowledge, reasoning the fused knowledge, obtaining implicit knowledge (detailed description will be given below to the specific example of reasoning with reference to FIG. 3), calculating the degree of confidence, and storing the implicit knowledge, of which the degree of confidence exceeds the threshold, into the MKB 205. Thus, the autonomous learning ability of the MKB 205 is enhanced, and more valuable knowledge is mined.

On the basis of the created or updated MKB 205, an index is generated and used by the processor 204. In general, under the guidance of an ontology base, at least one of recognition of entities inputted by the user, index generation, knowledge extraction from the semi-structured data and the unstructured data, integration of heterogeneous data, or reasoning supplement of data is implemented. The ontology base may be generated by utilization of various ontology generation systems. It should be noted that: as shown in FIG. 2, the ontology generation system and the ontology base do not need to reside in the intelligent triage server 202 and may be communicated and connected with the intelligent triage server for application. The MKB 205 is not necessarily disposed locally in the intelligent triage server 202, as long as the MKB can be called by the retrieval module 204. That is to say, the MKB 205 may be stored into other servers communicated and connected with the intelligent triage server 202.

For example, the intelligent triage server 202 may be implemented as various kinds of devices being provided with a processor and having enough operational capability, e.g., a centralized server or a distributed (cloud) server. Although description has been given here to the intelligent triage system 200 comprising the intelligent triage terminal 201 and the intelligent triage server 202 based on the MKB communicated and connected with the intelligent triage terminal, it should be noted that the intelligent triage terminal 201 and the intelligent triage server 202 may be respectively implemented as mutually independent and remote devices, may be respectively used, manufactured and sold, and are not necessarily integrated together.

FIG. 3 is a layered architecture diagram of an intelligent triage system 200 based on an MKB provided by the third embodiment of the present disclosure. As illustrated in FIG. 3, the intelligent triage system 200 comprises a data layer, a model layer and an application layer. Detailed description will be given below to the steps executed by the layers respectively.

1. Data layer: knowledge extraction and knowledge fusion from data sources such as medical guides, medical literature, electronic case history and medical web pages. The computer is adopted to read and understand massive medical literature, and extract tens of thousands of writing rules summarized for certain knowledge. As for the “disease-symptom” relationship, the ways described in the literature are: “the symptoms of (X disease) include (Y symptom)”, “the clinical manifestation of (X disease) is (Y symptom)”, “(X disease) tends to cause (Y symptom) and other symptoms”, etc. The following two types of data are analyzed and extracted: semi-structured data (e.g., the electronic case history) and unstructured text data (e.g., the medical literature). For example, the entities, the attributes and the relationships are extracted from the semi-structured or unstructured data by natural language processing technology; subsequently, the extracted knowledge is fused (mainly including entity alignment and linking, attribute value decision and determination, and relationship building); and implicit knowledge is found by the reasoning of the fused knowledge. For instance, when there are two pieces of known knowledge “there is calcium in broccoli” and “calcium can prevent osteoporosis”, implicit knowledge “broccoli can prevent osteoporosis” can be obtained. An MKB is created on the basis of the above steps.

Knowledge extraction generally includes entity extraction, relationship extraction and attribute extraction, For example further including quality evaluation. For instance, the degree of confidence of the extracted knowledge is evaluated, and only the relatively reliable knowledge is stored into the MKB 205 for expansion. For example, entity/attribute extraction adopts automatic extraction manner, mainly including supervised algorithm (e.g., SVM, MaxEnt and CRF), unsupervised algorithm, semi-supervised algorithm (e.g., bootstrapping) and distant supervision; and relationship extraction mainly adopts supervised method, namely utilizing a labeled data training model for classification.

Description will be given below to the specific application of the bootstrapping algorithm for entity/attribute extraction:

Input: seed examples of certain given category

Output: other examples of given category

Steps:

1. Expanding the inputted seed examples into a seed set;

2. Extracting templates from the literature according to the seed examples in the seed set;

3. Scoring and sorting the extracted templates, and expanding the prior n templates into a template set, in which n is a natural number;

4. Extracting examples from the literature according to the templates in the template set;

5. Scoring and sorting the extracted examples, and expanding the prior m examples into a seed set, in which m is a natural number; and

6. Repeating the steps 2-5 until the specific stop condition is reached or the example set no longer changes.

For instance, giving the disease name “diverticulitis” corresponding to “having blood in one's stool”, seed set S={ }, template set P={ }:

S1: allowing S={diverticulitis}, P={ };

S2: obtaining a template “* patients often have constipation” from a sentence in the literature “diverticulitis patients often have constipation”, in which * is a wildcard; obtaining a template “abdominal pain with blood in the stool should consider peptic ulcer disease or *” from a sentence “abdominal pain with blood in the stool should consider peptic ulcer disease or diverticulitis”; obtaining many templates from large data of the literature;

S3: scoring the above templates, in which the scoring methods include extracting features and using the supervised method to create a regression model for score prediction, or adopting bipartite graphs and using graph propagation method for scoring;

S4: supposing that the template “* patients often have constipation” is expanded into the template set P={“* patients often have constipation”, . . . } in the step S3, obtaining an example “hemorrhoid” from a sentence in the literature “hemorrhoid patients often have constipation” on the basis of the template; obtaining many examples by the method;

S5: scoring the examples obtained in the step S4, in which the scoring method is similar to the above scoring method for the templates; expanding the prior m examples with high score into the seed set; and

S6: repeating the above steps, and outputting the finally obtained seed set.

Knowledge fusion includes two aspects: data aspect and semantic aspect. Illustration has been given to how to fuse the knowledge in the description with reference to FIGS. 2 and 4, so no further description will be given here.

2. Model layer: the layer includes the foregoing intelligent triage server 202. Illustration has been given to the calling of the MKB 205 by the intelligent triage server 202 in the description with reference to FIG. 2, so no further description will be given here.

3. Application layer: the application layer includes the intelligent triage terminal 201; an interface of the intelligent triage terminal 201 provides an interactive page; the intelligent triage terminal 201 is connected to the intelligent triage server 202 based on the MKB 205 and configured to receive the retrieval request of the user and transmit the retrieval request to the intelligent triage server 202, and subsequently receive the retrieval results obtained by the intelligent triage server 202 from the MKB 205 on the basis of the retrieval request, and provide the retrieval results to the user; both the retrieval request of the user and the retrieval result adopt the form of natural language, so that the user can conveniently adopt natural language for inquiry and can also easily interpret the displayed analysis result in natural language. The user can adopt various methods to input the retrieval request, including keyboard input, handwriting input, check in choice box or a combination thereof. For example, the retrieval request includes questions checked in choice box or handwritten by the user to the intelligent triage terminal 201. Thus, the user asks a question and makes an inquiry through the intelligent triage terminal 201, may also obtain the final retrieval results, including disease suggestion, probability, recommended department and the like, from the intelligent triage server 202 through the selection of a series of symptoms displayed by the intelligent triage terminal 201, and display the retrieval results to the user on the interface in natural language.

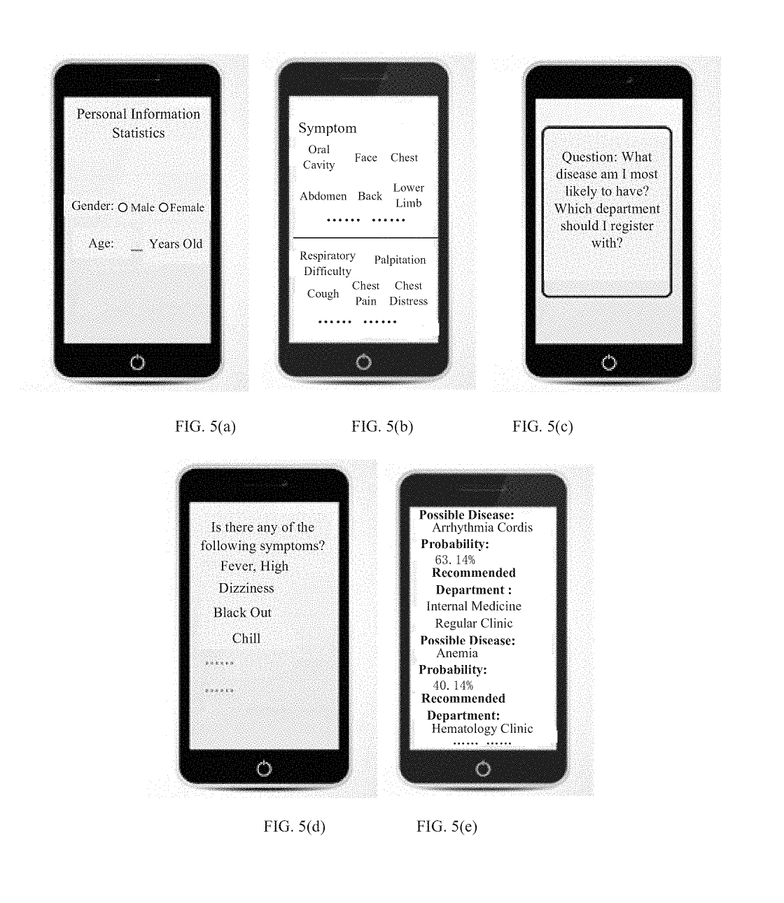

FIG. 5 is a schematic diagram of an intelligent triage terminal 201 based on an MKB provided by the fifth embodiment of the present disclosure. For example, the intelligent triage terminal 201 may be implemented as various devices such as various portable smart devices, including PDA, mobile phone, tablet PC, notebook computer and the like, and may also be implemented as non-portable smart devices such as tablet PC. The intelligent triage terminal may include a processor and a memory, the memory is for storing computer program instructions, the computer program instructions, when being executed by the processor, perform the following functions of the intelligent triage terminal.

Description is given in FIG. 5 to an interactive interface of the intelligent triage terminal by taking the intelligent triage terminal 201 implemented as a mobile phone as an example.

The intelligent triage terminal 201 is connected to the intelligent triage server 202 based on the MKB 205 and configured to receive a retrieval request inputted by the user and transmit the retrieval request to the intelligent triage server 202; the retrieval request may include personal information (as shown in FIG. 5(a)), symptom information (as shown in FIG. 5(b)) and question (as shown in FIG. 5(c)); subsequently, the intelligent triage terminal receives retrieval results obtained by the intelligent triage server 202 from the MKB 205 on the basis of the retrieval request, and displays the retrieval results to the user (as shown in FIG. 5(e)); and it can be seen that both the retrieval request of the user and the retrieval results adopt the form of natural language. The retrieval request includes questions checked in choice box or typewritten by the user to the intelligent triage terminal. For instance, FIG. 5(c) shows the questions inputted by the user “What disease am I most likely to have? Which department should I register with?”. Choice boxes of the symptom information are provided on the intelligent triage terminal 201. When the choice box of the symptom information is selected by the user, the intelligent triage terminal transmits corresponding symptom information to the intelligent triage server 202. For instance, as shown in FIG. 5(b), when a choice box of symptom information “palpitation” is selected by the user, the symptom information “palpitation” is transmitted to the intelligent triage server 202. For example, as shown in FIG. 5(b), the choice boxes include choice boxes of physiological parts and choice boxes of corresponding symptom information. When a choice box of a physiological part “chest” is selected by the user, choice boxes of corresponding symptom information are displayed, including “respiratory difficulty, palpitation, cough, chest pain, chest distress”. More For example, when the choice box of the symptom information, e.g., “palpitation”, is selected by the user, the intelligent triage terminal 201 provides choice boxes of symptom information relevant to the selected symptom information on the interface, e.g., “fever, high fever”, “dizziness”, “black out” and “chill”, for the user to select, so as to more comprehensively reflect all the symptom information of the user, and then the intelligent triage server 202 can provide more accurate retrieval results on this basis. The user For example adopts the choice boxes to select the symptom information. As ordinary users have different and sometimes non-standard expressions of symptoms, the intelligent triage server 202 is more difficult in the entity recognition and type analysis of the symptom information. When the choice boxes are adopted for the selection of the symptom information, in particular composite choice boxes including choice boxes of physiologic parts and choice boxes of corresponding symptom information, the user can be prompted to more accurately, regularly and comprehensively input the symptom information, so as to reduce the workload of the intelligent triage server 202 in the entity recognition and type analysis of the symptom information. The questions of the user “What disease am I most likely to have? Which department should I register with?” are transmitted to the intelligent triage server 202, so that the intelligent triage server can classify the questions, for instance, pre-diagnosis type and triage type, by utilization of natural language processing technology, acquire the true intention of the user, obtain precise retrieval results from the MKB 205, and provide the retrieval results to the intelligent triage terminal 201, as shown in FIG. 5(e).

For example, the intelligent triage terminal 201 may display the retrieval results to the user in sequence, and the displayed order is specified by the user. When the retrieval request at least belongs to the pre-diagnosis type, more For example, the retrieval results may be sorted according to the probability of possible diseases from high to low, so that the user can intuitively obtain those items of most probable diseases on a limited display region of the terminal; and in the items of the diseases, possible disease, probability and recommended department and the like may be displayed in sequence according to the user's intention, as shown in FIG. 5(e).

The foregoing embodiments are only the illustrative embodiments of the present disclosure and not intended to limit the present disclosure. The scopes of the disclosure are defined by the claims. The skilled in the art may make any change, or equivalent replacement to the disclosure in the essential scope of this disclosure, these changes and replacements shall be regarded as fall within the scope of protection of the present disclosure.

The present application claims the priority of the Chinese Patent Application No. 201710642039.7 filed on Jul. 31, 2017, which is incorporated herein in its entirety by reference as part of the disclosure of the present application.

1. An intelligent triage server based on a medical knowledge base (MKB), comprising: a processor and a memory,
the memory is for storing computer program instructions,
wherein, the computer program instructions, when being executed by the processor, perform the processes of:
receiving a retrieval request from an intelligent triage terminal;
performing entity recognition and type analysis on the retrieval request;
retrieving one or more pieces of knowledge by utilization of the index of the MKB according to the entities recognized and the types analyzed;
sorting the one or more pieces of knowledge retrieved, and regarding the prior knowledge as analysis results; and
transmitting the analysis results obtained to the intelligent triage terminal.

2. The intelligent triage server based on the MKB according to claim 1, wherein, sorting the one or more pieces of knowledge retrieved comprises:
sorting the one or more pieces of knowledge retrieved according to the degree of confidence from high to low.

3. The intelligent triage server based on the MKB according to claim 1, wherein, performing entity recognition and type analysis on the retrieval request comprises:
performing entity recognition via at least one method or a combination of hidden Markov model (HMM), maximum entropy model (MaxEnt), support vector machine (SVM) and conditional random field (CRF).

4. The intelligent triage server based on the MKB according to claim 1, wherein, performing entity recognition and type analysis on the retrieval request comprises: performing entity recognition via bootstrapping method.

5. The intelligent triage server based on the MKB according to claim 1, wherein, performing entity recognition and type analysis on the retrieval request comprises:
performing type analysis via at least one classifier or a combination of decision tree, random forest, logistic regression, gradient boost and SVM.

6. The intelligent triage server based on the MKB according to claim 1, wherein the recognized entities comprise at least one of the follows: symptom entity, time entity, disease entity, personal information entity and medical site entity.

7. The intelligent triage server based on the MKB according to claim 1, wherein the types comprise at least one of the follows:
pre-diagnosis type, triage type and healthcare type.

8. The intelligent triage server based on the MKB according to claim 1, wherein the MKB comprises structural data.

9. The intelligent triage server based on the MKB according to claim 8, wherein, the computer program instructions, when being executed by the processor, further perform the processes of:
creating or updating the MKB.

10. The intelligent triage server based on the MKB according to claim 9, wherein, creating or updating the MKB comprises:
extracting entities, attributes, and relationships, from semi-structured data and unstructured data in a data source, as knowledge;
fusing the extracted knowledge; and
calculating the degree of confidence of the fused knowledge, and storing the knowledge, of which the degree of confidence exceeds the threshold, into the MKB.

11. The intelligent triage server based on the MKB according to claim 9, wherein creating or updating the MKB further comprises:
after fusing the extracted knowledge,
obtaining implicit knowledge by the reasoning of the fused knowledge; and
calculating the degree of confidence of the obtained implicit knowledge, and storing the implicit knowledge, of which the degree of confidence exceeds the threshold, into the MKB as well.

12. The intelligent triage server based on the MKB according to claim 10, wherein extracting entities, attributes from semi-structured data and unstructured data comprises:
extracting entities, attributes from semi-structured data and unstructured data by any one of supervised algorithm, semi-supervised algorithm, unsupervised algorithm and distant supervision algorithm.

13. The intelligent triage server based on the MKB according to claim 10, wherein extracting relationships from semi-structured data and unstructured data comprises:
extracting relationships from the semi-structured data and the unstructured data by supervised algorithm.

14. The intelligent triage server based on the MKB according to claim 10, wherein fusing the extracted knowledge comprises:
integrating the knowledge extracted from the semi-structured data and the unstructured data, in which
the integration includes entity alignment, attribute value decision and relationship building.

15. A intelligent triage terminal, which is connected to the intelligent triage server based on the MKB, comprising: a processor and a memory,
the memory is for storing computer program instructions,
wherein, the computer program instructions, when being executed by the processor, perform the processes of:
receiving a retrieval request inputted by an user;
transmitting the retrieval request to the intelligent triage server;
receiving retrieval results obtained by the intelligent triage server from the MKB on the basis of the retrieval request;
displaying the retrieval results to the user.

16. The intelligent triage terminal according to claim 15, wherein,
displaying the retrieval results to the user comprises:
displaying the retrieval results to the user based on a sequence specified by the user.

17. The intelligent triage terminal according to claim 15, wherein,
the retrieval results is sorted according to the probability of possible diseases from high to low by the intelligent triage terminal, items of probable diseases is displayed on a display region of the intelligent triage terminal.

18. The intelligent triage terminal according to claim 15, wherein, both of the retrieval request of the user and the retrieval results comprises a form of natural language.

19. A intelligent triage system, comprising:
the intelligent triage server based on the MKB according to claim 1 and an intelligent triage terminal,
wherein, the intelligent triage terminal is connected to the intelligent triage server based on the MKB, comprising: a processor and a memory,
the memory is for storing computer program instructions,
wherein, the computer program instructions, when being executed by the processor, perform the processes of:
receiving a retrieval request inputted by an user;
transmitting the retrieval request to the intelligent triage server;
receiving retrieval results obtained by the intelligent triage server from the MKB on the basis of the retrieval request;
displaying the retrieval results to the user.

20. A intelligent triage system, comprising:
the intelligent triage server based on the MKB according to claim 2 and an intelligent triage terminal,
wherein, the intelligent triage terminal is connected to the intelligent triage server based on the MKB, comprising: a processor and a memory,
the memory is for storing computer program instructions,
wherein, the computer program instructions, when being executed by the processor, perform the processes of:
receiving a retrieval request inputted by an user;
transmitting the retrieval request to the intelligent triage server;
receiving retrieval results obtained by the intelligent triage server from the MKB on the basis of the retrieval request;
displaying the retrieval results to the user.
